(19) United States
(12) Patent Application Publication
(10) Pub. No.: US 2010/0041972 A1
(43) Pub. Date: Feb. 18, 2010

(54) APPARATUS AND METHODS

(75) Inventor: Roger Mason, Cambridge (GB)

Correspondence Address: WOLF GREENFIELD & SACKS, P.C., 600 ATLANTIC AVENUE, BOSTON, MA, 02210-2206, US

(73) Assignee: Lectus Therapeutics Limited, Cambridge, Cambridgeshire (GB)

(21) Appl. No.: 12/309,084

(22) PCT Filed: Jul. 9, 2007
(86) PCT No.: PCT/GB2007/050390

§ 371 Date: Oct. 6, 2009

(30) Foreign Application Priority Data
Jul. 7, 2006 (GB) 0613500.8

(51) Int. Cl. A61B 5/0492; A61N 1/04; A61N 1/05; A61B 5/04; H01R 43/00; C12Q 1/02; B44C 1/22; C12M 1/34; G01R 27/00

(52) U.S. Cl. 600/372; 607/148; 607/116; 600/546; 29/855; 435/29; 216/13; 435/287.1; 324/649

(57) ABSTRACT

The invention relates to a multi-electrode array comprising a plurality of electrodes, the electrodes being spaced apart from each other by spacer means, the electrodes being secured to the spacer means, the spacer means being encapsulated within a housing. The invention also relates to an implantable device comprising a multi-electrode array of the invention, a method of manufacturing electrodes for use in an array of the invention and a method for manufacturing a multi-electrode array of the invention. The invention also relates to a method for monitoring the effect of a test substance on a biological tissue using a multi-electrode array of the invention.

### TECHNICAL FIELD

The present invention relates to multi-electrode arrays (MEAs), methods for manufacture of MEAs, implantable devices comprising MEAs and to the use of MEAs in electrophysiological methods for monitoring tissues, tissue samples or cultures, in particular for monitoring the response of the tissue samples or cultures to test compounds.

### BACKGROUND TO THE INVENTION

Multi-electrode arrays in conventional substrate-based format may comprises “2-D” arrays of electrodes, in the form of disks or 70 μm high spikes, printed using photolithographic techniques, spaced relative to each other at equidistant addresses on a substrate, which is usually in the form of a shallow dish.

Assays using MEAs are performed in plate format, such that the tissue under examination is placed on top of an array of disk or 70 μm high electrodes for measurement of electrical signals. MEAs have been used to stimulate and record extracellular electrical activity of excitable biological tissue from as many as 60 recording sites simultaneously. In most cases, single unit activity and/or slow field potentials are recorded in preparations such as dissociated cell cultures (neurons, heart muscle cells) and organotypic or acute tissue slices (brain, spinal cord, retina, etc.).

A disadvantage of these MEA formats is that they are not suited for use with contractile tissue such as smooth muscle or heart, as such tissue is contoured, may contract during recording, does not sit well on the electrodes and needs to be compressed or ‘sucked down’ to achieve good contact, thereby damaging the tissue and potentially giving anomalous results.

An additional disadvantage is that the prior art MEAs are not readily repositionable; to reposition the electrodes the tissue must moved on the substrate. The electrodes cannot be adjusted depthwise, thus cannot be configured to contact a single layer within the tissue.

One form of MEA has electrodes which are spaced by threading them through two electron microscope (e.m.) grids to form an array, the electrodes are then glued to the e.m. grid. There is no encapsulation of the electrodes or e.m. grid; effectively this array is a bundle of wires in which the filaments are spaced by the e.m. grid. In use the ends of the electrodes in the prior art device are each individually attached to a manipulator; as the device is not in unit form, it is not configured to be readily transferred and held on a manipulator or microdrive unit. This type of MEA has not been used in tissue assays and although it has been used as a short term, acute implant in a whole animal context, it has not and could not be used as a chronic implant.

In known MEAs the tips of the electrodes are in planar configuration, thus they cannot be configured to the contours of a tissue, either at the surface of the tissue, or penetrating into tissue to contact a layer or layers within the tissue. These MEAs detect only electrical activity; they can not detect movement of the tissue, i.e. contractions. Movement in tissue cannot be assessed using dish format MEAs, nor using MEA with electrodes formed from fused quartz filaments, as these lack the necessary resilience to act as mechanical transducers to sense movement. Thus, known MEAs have not been used to detect and correlate electrical signals within a contractile tissue with the associated movement of the tissue.

There is a desire to develop MEA assays for use in drug discovery to provide information on biological response of tissue to drug substances.

### DISCLOSURE OF THE INVENTION

The invention provides a multi-electrode array comprising a plurality of electrodes, the electrodes being spaced apart from each other by spacer means, the electrodes being secured to the spacer means, the spacer means being encapsulated within a housing.

A multi-electrode array in accordance with the invention will comprise at least two electrodes, in typical MEA formats of the invention 2, 4, 8, 16, 32, 64, 128, 256 or 512 electrodes are present in the array. The number of electrodes in the MEA may be selected to correspond to the number of recording channels in the detection system employed in electrophysiological assays.

The spacing of the electrodes in the array depends on the proposed use of the MEA. For electrophysiological assays, spacing of electrodes in the MEA is generally in the range of from about 80 μm to about 400 μm, suitably from about 100 μm to about 300 μm, e.g. 250 μm. For implantable devices, a greater spacing is usually desired, for example in the range of from about 250 μm to about 1000 μm, e.g. 500 μm.

In multi-electrode arrays of the invention the spacer means and housing can be unitary, thus the spacer means can form an integral part of the housing; for example the spacer means may be channels in a block, the block forming the housing.

The electrodes employed in MEAs of the invention will typically have a diameter in the range of from about 30 μm to about 200 μm, preferably from about 40 μm to about 150 μm, e.g. 125 μm.

The electrodes can be made from tungsten, stainless steel, platinum, platinum/iridium, carbon fibres, conductive nanotubes, carbon nanotubes, an Elgiloy® alloy (Elgiloy Specialty Metals, Elgin, IL60123, USA), or a conductive polymer (e.g., polythiophene or oxidised polypyrrole).

Metal electrodes, such as wires, are particularly useful in MEAs of the invention. Metal electrodes with shape memory, e.g. formed from stainless spring steel, may be used; such electrodes are particularly useful for MEAs that are to be incorporated into implantable devices.

MEAs of the invention may be constructed such that at least one of the electrodes is hollow. Hollow electrodes are conductive tubes, for example, formed from polyimide, carbon, e.g. carbon nanotubes, or from metal, e.g. fine steel capillary tubes.

MEAs with one or more hollow electrodes may be incorporated into implantable devices, which may be acute or chronic implants; for example to enable local delivery of a substance, such as a drug substance, or for sampling fluid from the site at which the MEA contacts the tissue. The hollow electrode may be connected to a drug reservoir, from which the drug substance may be administered; administration of the drug can be controlled via electrical signals sensed by the MEA, such that when a particular electrical signal is detected by the electrodes of the MEA, administration of the drug from the reservoir is triggered. Such chronic implants are envisaged for use, for example, to treat overactive bladder with or without incontinence, urge incontinence, mixed incontinence, epilepsy, Parkinson's disease, pain, cardiac arrhythmias, anxiety, depression or any other condition which requires precise, local administration of medication. Local drug administration from a chronic implant is particularly useful for drugs that are difficult or impossible to administer systemically.

The spacer means can be plurality of electron microscopy grids, e.g. 2, 3, or 4 e.m. grids; a plurality of fine mesh patches e.g. 2, 3, or 4, mesh patches, for example steel mesh (e.g. of a spacing as described above), or a block containing channels (e.g. of a spacing as described above). The gaps or channels in the spacer means are dimensioned such that the electrodes can be threaded into the gap or channel.

In multi-electrode arrays of the invention, the electrodes can be spaced in a regular pattern or a regular repeating pattern. Electrodes can be arranged in a patterns such that the electrodes within the pattern are set at different lengths, e.g. for penetration of different layers of tissue. MEAs may be prepared using multiple blocks of the same pattern or of different patterns of electrodes. The electrodes in MEAs of the invention may be spaced so that they are equidistant from each other. Electrodes may be positioned in MEA, for example, in a hexagonal or radial pattern (with a central electrode, which may be hollow), or a square pattern.

The spacer means and/or housing can be formed from a mouldable material, e.g. an acrylic resin, plastics, thermoplastics, thermoset, or rubber material. For implantable devices in particular, the spacer and/or housing may be formed from a flexible material to enable the MEA to conform to the contours of the tissue at the implant site. For implants, the housing is most preferably a biocompatible material, such as a biocompatible thermoplastics material.

The electrodes can be permanently secured to the spacer means. However, preferably, the electrodes are releasably secured to the spacer means, such that the length of the electrode can be adjusted and so that when an individual electrode is damaged it can be replaced. The ability to adjust the length of the electrodes is useful when the MEAs are to be used in electrophysiological assays, as this allows the electrodes to be positioned to contact the surface of contoured tissue or to contact a single layer within tissue. Electrodes can be glued to the spacer means, e.g. using a dissolvable or softenable adhesive for releasably securing the electrodes; alternatively, fixing means, such as releasable clamps, can be used to secure the electrodes to the spacer means.

In multi-electrode arrays of the invention the electrodes can be secured permanently so that the tips are in planar or non-planar configuration, or releasably secured so that the tips can be adjusted to a planar or non-planar configuration.

Each electrode will generally comprise a connector, distal to the sensing tip of the electrode. The connector can be used to couple the electrodes of the MEA to a multichannel electrophysiological data acquisition system; many such systems are commercially available (e.g. Multichannel Systems GmbH, Germany).

The connector may be a push-fit connector, which is preferably releasable to enable the electrodes readily to be attached and detached.

MEA comprised in implants may be accessed remotely using telemetry.

The electrodes are typically provided with an electrical insulating layer or jacket along at least a part of the length of the electrode, the tip of the electrode being exposed to enable electrical contact in use. Suitable insulating materials include epoxylite resin, glass, fused quartz, polyimide, Parylene-C™, urethan, diamond and Formvar™.

In multi-electrode arrays of the invention, the electrodes are multifunctional in that they can detect electrical signals, stimulate the tissue electrically, or can act as mechanical transducers.

MEAs of the invention may be incorporated into devices for implantation, e.g. deep brain implants for stimulation of the sub-thalamic nucleus to control Parkinson's tremor, or other CNS targets for epilepsy and pain; or implantable defibrillators. Such implantable devices may incorporate metal electrodes with shape memory, conferred by curvature of the electrodes, this aspect is useful for accurate positioning of electrodes in the target tissue; this represents a significant improvement over currently available devices.

As described herein, implants may comprise MEAs with one or more hollow electrodes that can be used for local site-specific drug delivery. Positioning of electrodes and/or drug delivery can be controlled by electrical signals sensed by the implant device. During implantation the implant may sense and report electrical signal and correct positioning of the implant can be detected when the desired effect on electrical signal is reported. MEA of the invention can be incorporated into implantable defibrillators and correct positioning of the implant can be detected when the desired pattern of cardiac activity is detected. MEA of the invention can be incorporated into implants destined for the subthalamic nucleus to control the tremor associated with Parkinson's disease. The MEA can also be incorporated into implants for control of epilepsy.

### Assay Methods

MEAs of the invention can be incorporated into conventional electrophysiology assay apparatus to monitor the electrical signals in excitable tissue.

This invention provides the use of a MEA according to the invention in an electrophysiological assay, for example an electrophysiological assay to assess the effect of a test substance, e.g. a test compound, on a tissue sample or tissue culture. These assays may be used to identify potential therapeutic agents, to assess drug side effects, to identify toxic substances, such as environmental pollutants, nerve agents and the like.

An MEA of the invention may also be incorporated into a sensing device for environmental monitoring, to detect, pollutants or toxins and similar.

In one aspect the invention further provides a method for monitoring the effect of a test substance on a biological tissue comprising:
(a) providing a biological tissue in a medium,
(b) contacting the electrode tips of a MEA, in particular a MEA of the invention, with the tissue,
(c) recording the electrical signal detected by one, some, or all of the electrodes,
(d) optionally recording movement in the tissue by recording mechanical transduction of the electrodes (e.g. an electrical signal corresponding to this) and/or using optical means,
(e) exposing the tissue to a test substance,
(f) recording the electrical signal detected by the electrode(s),
(g) optionally recording the movement in the tissue by recording mechanical transduction of the electrode(s) and/or using optical means,
(h) comparing the results recorded in the absence and presence of the test substance.

MEAs of the invention are capable of acting as dual function detectors, in that they can detect not only electrical signals, such as muscle action potentials or neural activity (which are detected as high frequency electrical signals), or local field potentials, e.g. in a brain slice or whole brain, (which are detected as lower frequency electrical signals) but also, the electrodes can act as mechanical force transducers that can detect movement in contractile tissue, e.g. on muscle contraction. Transducers translate the movement in a tissue to electrical energy because when the elements are subjected to mechanical stress a voltage is produced (typically detected as a low frequency signal, e.g. less than 10 Hz). Thus electrical activity and movement within tissue can be detected simultaneously and these data can be correlated.

Detection of these electrical signals can be combined with use of an optical device to monitor and/or record movement in tissue, these data can be correlated.

Any biological tissue sample or culture may be used in methods of the invention. The biological tissue may comprise excitable and/or non-excitable tissue components. The methods may simply be electrophysiological assays, suitable for assessing the effect of test substances on tissues such as brain tissue samples (e.g. brain slice, whole brain) or brain tissue cultures (e.g. organotypic/neural cultures, spinal cord preparations). Additionally, because the MEA of the invention can be used to detect both electrical signals and movement of the tissue, they are particularly useful in methods for assessing the effects of test substances on contractile tissue samples, e.g. smooth muscle (e.g. bladder, urethral, ureteral, vas deferens, aortic, vascular, mesenteric, airway smooth muscle, tracheal, bronchial, pulmonary, renal artery, venous, arterial, gastrointestinal, uterine, pupillary sphincter, or lymphatic); cardiac muscle (e.g. ventricular, atrial or papillary), or skeletal muscle. The methods may also be performed on distributed cell cultures, such as muscle cell cultures (e.g. cardiac myocyte cultures) or dorsal root ganglia.

Tissue for use in assays of the invention may be human tissue obtained as post-mortem samples (provided the tissue itself is still live) or by biopsy; alternatively tissue from non-human animals may be used.

The tissue may be normal tissue, or may be tissue associated with a particular pathology, e.g. hippocampus from an animal displaying epileptiform activity.

Methods for sampling, preparation and maintenance of tissue samples, and for tissue culture, e.g. organotypic or distributed cell culture, are well known in the art. Similarly, media for tissue samples and for tissue culture are well known in the art, the choice of media used in methods of the invention being dictated by the tissue sample or culture used. Krebs buffer can be used for tissue samples such as bladder preparations. The composition of media used to replace physiological fluids such as cerebrospinal fluid is also well known.

In methods of the invention, tissue is placed or secured in a tissue chamber and bathed in a suitable medium, such as a tissue culture medium, which may be a defined medium, or other physiological solution to which a test substance is introduced. Assays in accordance with the invention are particularly useful when only small amounts of test substances are available, as they can be set up so that the medium containing the test substance is collected or recirculated. The medium may be sampled at time points during the assay and analysed, e.g. to detect metabolites, degradation products, metabolites of the test compound, or secondary messenger substances released from the tissue in response to the test substance.

In addition to assessing the effect of a test substance as a potential therapeutic, methods of the invention are useful to detect possible side effects such as unusual neuronal or muscular activity.

The electrodes of the MEA device are contacted with the tissue so that they are positioned on or penetrating into the tissue.

In MEAs of the invention the electrodes can be positioned in planar fashion, but can also be adjusted so that the electrodes are not planar, thereby allowing the electrodes to be configured to the contours of a tissue, either at the surface of a tissue, or penetrating into tissue to contact a layer or layers within the tissue. MEAs of the invention are unique in that they permit investigation of the tissue without compression damage and allow the electrodes to contact the same layer or different layers of tissue as desired. MEAs of the invention are also unique in that they can penetrate through dead tissue layers on the surface of tissue slices in a controlled fashion. MEAs in accordance with the invention are such that they can be placed on top of the tissue, and can be readily repositioned on the tissue, or penetrating into the tissue.

Detection of high frequency signals provides data on electrical activity within the tissue, detection of low frequency signals (resulting from the electrodes acting as mechanical transducers) provides an indication of movement in the tissue. The methods may incorporate use of an optical device to monitor movement of the tissue (e.g. tonic or major contraction). The effect of the test substance on electrical activity and associated motile response of the tissue can be monitored and assessed.

Methods of the invention can be used to measure electrical activity associated with the opening and closing of ion channels and their modulation by test compounds that act as pharmacological agents. In the case of muscle tissue, associated changes in contraction and tension and the frequency of contractile events can be monitored.

There are at least 400 ion channel genes encoding many different types of ion channels. Not all cells contain the same types of ion channels and precisely the same complement of ion channels. The behaviour of ion channels even within a class can be very different.

In general, at the resting membrane potential of cells, activation (opening) of sodium and calcium channels is excitatory; closing of potassium channels is excitatory. Thus for a muscle cell, activation of sodium and/or calcium channels would promote contraction; inhibition of potassium channels would have a similar effect. The same rationale applies to secretory cells where activation of sodium and/or calcium channels would promote secretion of, for example, a hormone. In the case of neuronal cells activation of sodium and/or calcium channels would promote neuronal firing and increase neuronal traffic; inhibition of potassium channels would have a similar effect. Thus, pharmacological modulation of the activity of ion channels can either promote or inhibit cellular activity and, therefore, tissue activity.

A test compound that blocks potassium channels would promote muscle contraction, neuronal firing or hormone secretion. A test compound that opens potassium channels would have the converse effect.

A test compound that blocks sodium channels would inhibit muscle contraction, neuronal firing or hormone secretion. A test compound that opens sodium channels would have the converse effect.

A test compound that blocks calcium channels would inhibit muscle contraction, neuronal firing or hormone secretion. A test compound that opens calcium channels would have the converse effect.

Methods of the invention are thus useful to detect substances such as compounds that modulate excitatory activity, e.g. compounds that suppress such activity or compounds that promote such activity. Compounds that suppress excitatory activity are useful in the treatment of conditions in which excitability is increased, such as overactive bladder, with or without incontinence, urge incontinence, mixed incontinence, pain (e.g. neuropathic pain), tinnitus or epilepsy. Compounds that promote excitatory activity are useful in conditions in which network excitability is reduced or compromised, such as Parkinson's disease, Huntingdon's disease or Alzheimer's disease. Methods of the invention are also useful to detect potential side effects of test compounds.

In methods of the invention the biological tissue can be subjected to excitation prior to exposure to the test compound. This is useful to detect compounds that suppress excitatory activity which may be useful in treatment of conditions of hyperexcitability, such as overactive bladder, with or without incontinence, urge incontinence, mixed incontinence, pain (e.g. neuropathic pain), tinnitus or epilepsy.

In methods of the invention each electrode of the MEA can be contacted with the same or single layer within a tissue of interest, e.g. a layer of muscle cells, which can be very advantageous. Neuronal tissue and smooth muscle is often composed of more than one layer of cells, each cell type performing a different function. To monitor response in a single layer or cells, i.e. in a single cell type, it is important that each of the electrodes in the array contacts the layer of cells of interest.

The signal may be detected from 2, 4, 8, 16, 32, 64, 128, 256, 512 electrodes. A minority of the electrodes may be used as the reference and as the ground electrodes to provide voltage measurements against which the other recordings are referred. For example, when using a 64 electrode MEA, four electrodes, e.g., the four corner electrodes, can be selected for use as the reference or ground electrodes. Alternatively, signals from all of the electrodes in the MEA can be recorded relative to reference and ground electrodes that are placed distant to the MEA

The electrical signals may be recorded and/or processed in a conventional manner. The signals may be recorded continuously or may be taken during suitable time segments, that is ‘binned’ e.g. every 10 or 20 seconds. Mechanical transduction of the electrodes may also be detected/recorded.

Additionally or alternatively, the movement of the tissue can be recorded using optical means such as a video device, e.g. a video microscope. Using image analysis software, positions on the image of the tissue may be marked and their movement traced.

Alternatively or additionally, a voltage sensitive dye or calcium sensitive dye may be included in the medium to facilitate optical means of movement detection/monitoring. Such dyes are readily available (e.g. from Molecular Probes, Oregon, USA). An increase in excitability produces a voltage change causing such dyes to fluoresce; this can be measured using commercially available equipment.

The results recorded in the presence and absence of the test substance may be compared. In some embodiments, correlating the electrical activity of the tissue with the movement of the tissue enables the spatio-temporal dynamics of the tissue to be assessed in the presence and absence of the test substance and enables the data to be compared to determine the effect of a test compound on the tissue. The data can be presented as chronotopograms or “movies”.

A method according to the invention may further comprise performing an intracellular electrophysiological assay, such as a patch clamp assay, on a cell or cells within the tissue. An intracelular assay may be performed to provide data on the intracellular status of cells within a tissue, which can be correlated with the extracellular activity detected using the MEA. Recordings made in this way provide details of the mechanism of action of the compound as an adjunct to the MEA data.

The invention further provides assay methods for monitoring the effect of a test substance, on a non-human animal comprising:
(a) providing an anaesthetised non-human animal,
(b) implanting the electrodes of an MEA in accordance with the invention at a desired site in the animal,
(c) recording electrical signals detected by the electrodes before and after administration of a test substance, and,
(d) comparing the electrical signals recorded before and after administration of the test substance.

The non-human animal is typically a rabbit, guinea pig, rat, mouse, non-human primate, dog or cat.

The implantation site can be any tissue of interest in the body of the non-human animal, such as smooth muscle (e.g. in the bladder), spinal cord, or brain.

The invention further provides a method of manufacturing metal electrodes comprising:
(a) providing a plurality of metal wires spaced apart from each other in a substantially parallel manner and secured to a mandrel,
(b) forming an electrical connection between the wires,
(c) attaching the electrical connection to a first port of an AC electrical power supply,
(d) repeatedly dipping the metal wires into and out of an etch solution to a predetermined depth, a low voltage AC current being provided between the wires and a conductor (e.g. a graphite rod or plate) within the etch solution, the conductor being attached to the second port of the AC electrical power supply,
(e) washing and drying the electrodes formed by electroetching the wires.

The metal wires can be Tungsten wires, which initially may have a diameter in the range of from about 100 μm to about 150 μm, e.g. 125 μm. A suitable etch solution for use with Tungsten is Levick's solution (e.g. 10 mol/l sodium nitrite and 6 mol/l potassium hydroxide).

Alternatively, the metal wires can be stainless steel wires and the etch solution can be potassium cyanide or concentrated sulphuric acid, optionally overlaid with mineral oil or Xylol.

The wires are generally spaced a few mm apart on the mandrel, in a substantially parallel manner. This is to ensure that each wire is etched to the same extent, and helps to provide a matched set of electrodes of similar diameter and electrical impedance.

The electrical connection between the wires can be made by using a mandrel having a conductive surface to which the wires are connected.

The wires are electroetched until the electrodes tips are the desired diameter, suitably about 1 μm. The conductor can be a rod, such as a graphite rod. The mandrel can be of circular cross section so that during the dipping procedure the electrodes are positioned radially around the rod.

The AC current applied is typically in the range of 4 to 8 V, e.g. 6V.

Generally, the electrodes will be dipped into the etch solution about 150 to 210, e.g. 180 times, to achieve the desired narrowing of the wires to form the electrodes.

Following etching the mandrel is disconnected and the electrodes are washed, e.g. by dipping in double distilled or reverse osmosis purified water, and dried, e.g. by air drying.

Following electroetching the electrodes can be provided with a layer of insulating material. This can be performed by dipping the electrodes into an insulating material in liquid form (e.g. with one slow dip); the thickness of insulating layer applied can be controlled by the speed at which the electrodes are withdrawn from insulating material, the electrodes can be dipped into the insulating material while still attached to the mandrel, this is useful to produce electrodes with a similar thickness of insulating coat. Alternatively the insulating layer can be applied in a vapour phase.

Insulating material that can be used to coat electrodes include epoxylite resin, glass, fused quartz, polyimide, Parylene-C™, urethan, diamond, and Formvar™.

The electrode tips are de-insulated before they are used. For heat labile insulating materials, this can be achieved by melting or burning away the insulating material using a heated platinum wire to expose a few hundreds of microns at the tip of the electrode. Alternatively the insulating material can be removed using a laser, or dissolved away.

The impedance of the electrodes can then be tested. Optionally a second etch can be performed on each electrode, usually individually, to form the electrode tip to a desired dimension and impedance.

The electrode tip can be conical, cylindrical or bullet shaped. Conical tips tend to have sharp projections that can “snag” tissue. In MEA of the invention it is preferred that the tip of the electrode is shaped like a bullet tip, as this shape has been found to be less damaging when inserted into and retracted from tissue. The tips may be shaped, for example, by etching, grinding or using a laser. Following a second etch the impedance of the electrodes may again be tested.

Optionally the electrode tip may be coated with gold or silver, using techniques well known in the art. Gold-coated electrode tips may optionally be coated with platinum black. Some metals such as tungsten may be cytotoxic during long-term implantation, and thus may be coated with gold to reduce cytotoxicity. Gold-coated electrode tips are more readily visible which facilitates positioning of the electrodes in tissue. Electrodes coated with platinum black are desirable as they have a lower impedance than uncoated electrodes and a higher signal to noise ratio.

The invention yet further provides a method of manufacturing a set of metal electrodes for use in a microarray, comprising connecting precursor electrode wires to a common electrical conductor and electrochemically etching the electrode wires together in an etching bath to create a narrowed tip on each of said wires, whereby said method creates a substantially matched set of electrodes.

Additionally the invention provides a matched set of electrodes produced by a method described herein.

The invention also provides a method of manufacturing a multi-electrode array of the invention comprising:
(a) providing a plurality of electrodes,
(b) spacing electrodes apart from each other by threading each electrode through a different gap or channel of a spacer means,
(c) securing the electrodes to the spacer means,
(d) encapsulating the spacer means within a housing.

In this aspect, MEAs as described herein can be prepared using a plurality of e.m. grids to space the electrodes from each other, or alternatively a plurality of fine mesh patches may be used.

The invention also provides a method of manufacturing a multi-electrode array of the invention comprising:
(a) providing a plurality of electrodes,
(b) spacing electrodes apart from each other by threading each electrode through a different channel of a spacer means and securing the electrodes to the spacer means, the spacer means forming an integral part of a housing that contains the spacer means.

In this alternative method for construction, electrodes are threaded through channels in a block to achieve array positioning of the electrodes. The block approach can be used to build up much larger arrays than are currently available. The block approach is advantageous in that the electrodes can readily be positioned in a non-planar tip configuration, furthermore, using a releasable securing means, such as dissolvable or softenable adhesive, or releasable clamp, electrodes can be easily removed and repositioned or replaced.

The electrodes are preferably matched such that they are of comparable diameter and impedance.

### FIGURES

FIG. 1 shows a 60 element multi-electrode array of the invention, termed a NeuroZond™ MEA. This MEA is constructed using 125 μM diameter tungsten electrodes coated with epoxylite, with exposed tips, spaced using ‘100 mesh’ square-packed configuration.

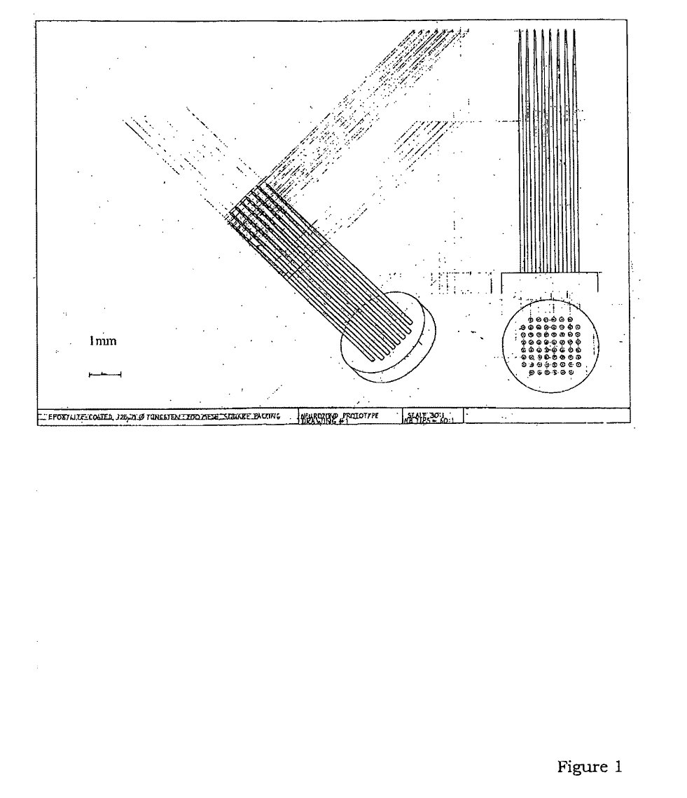

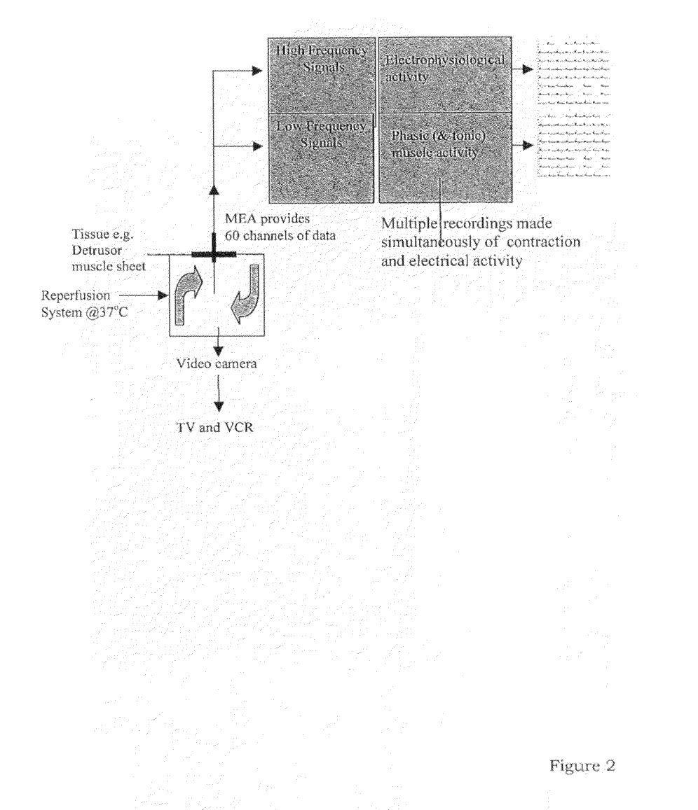

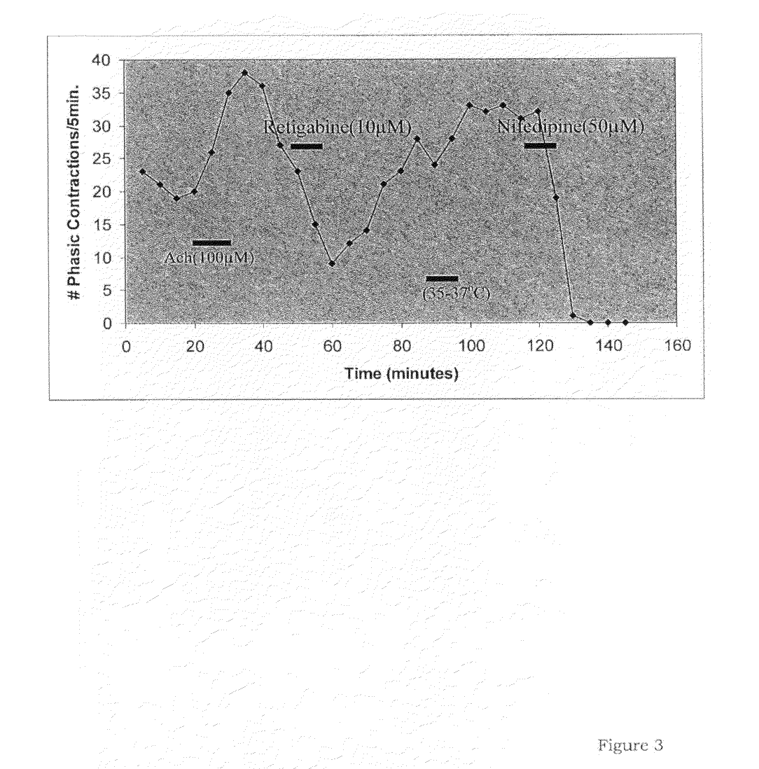

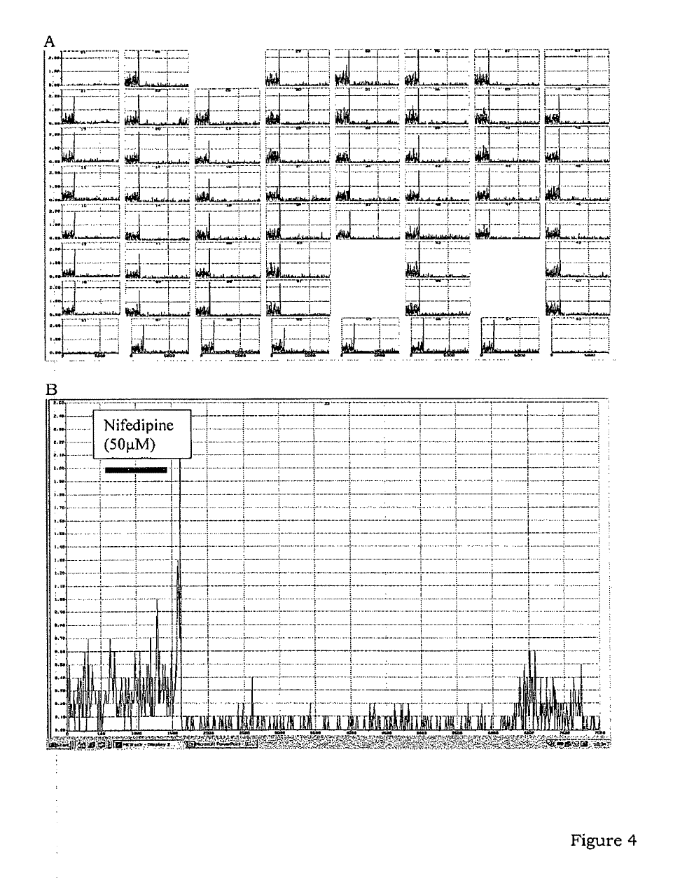

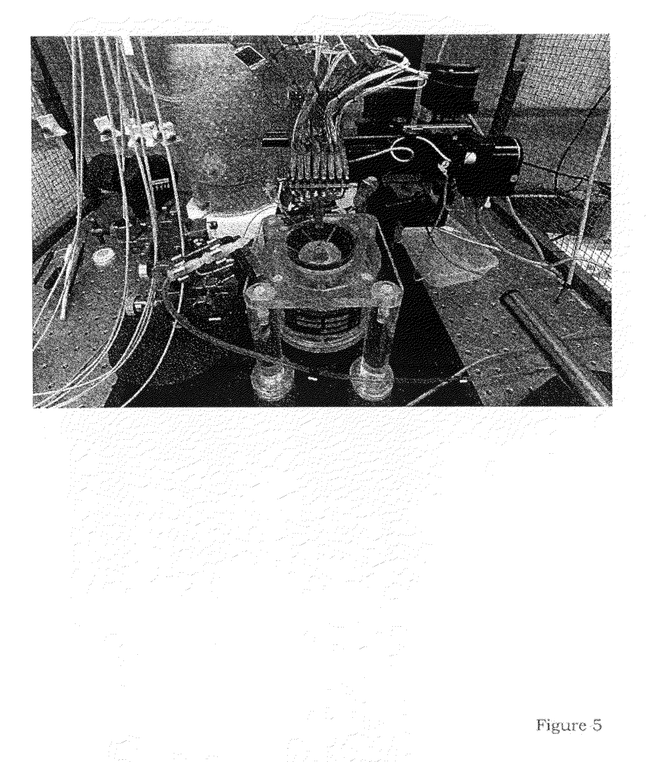

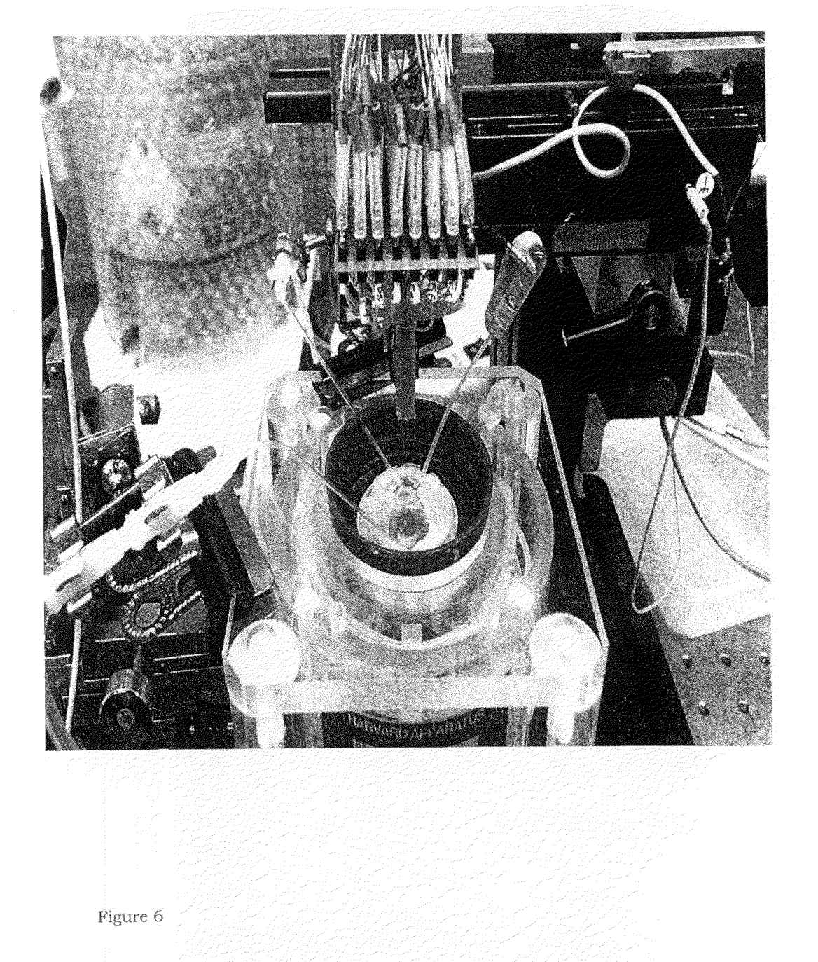

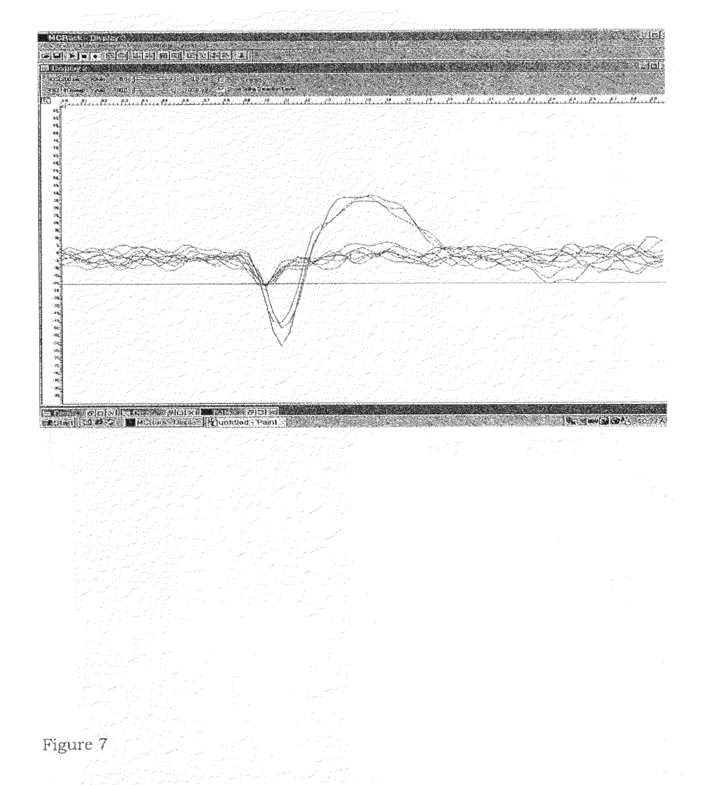

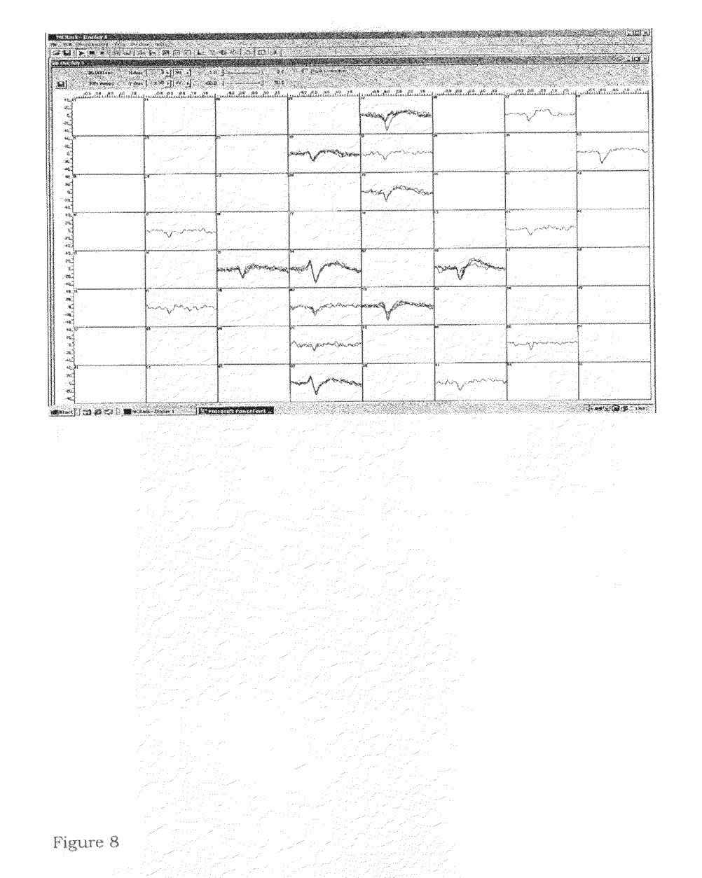

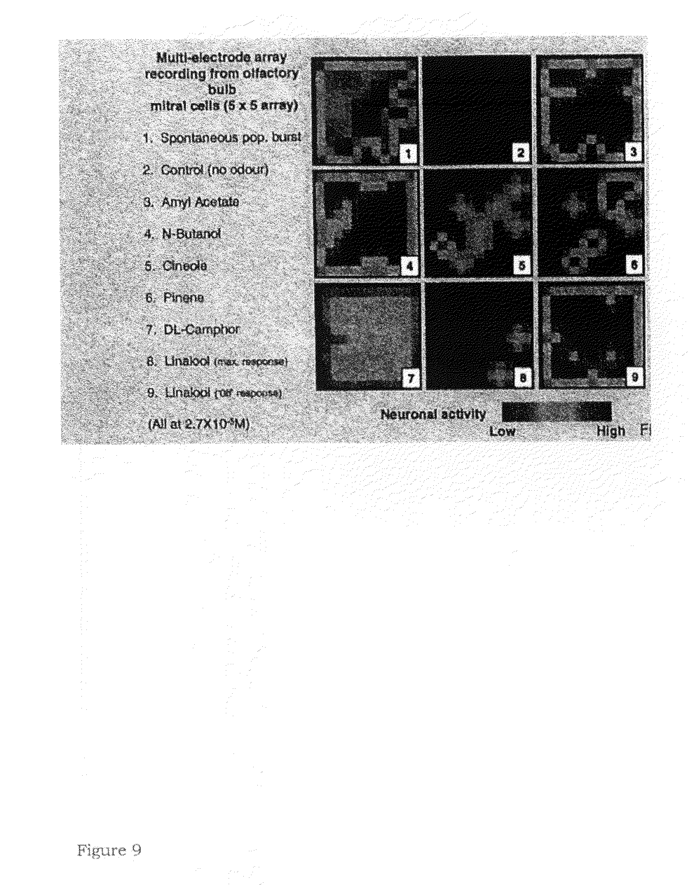

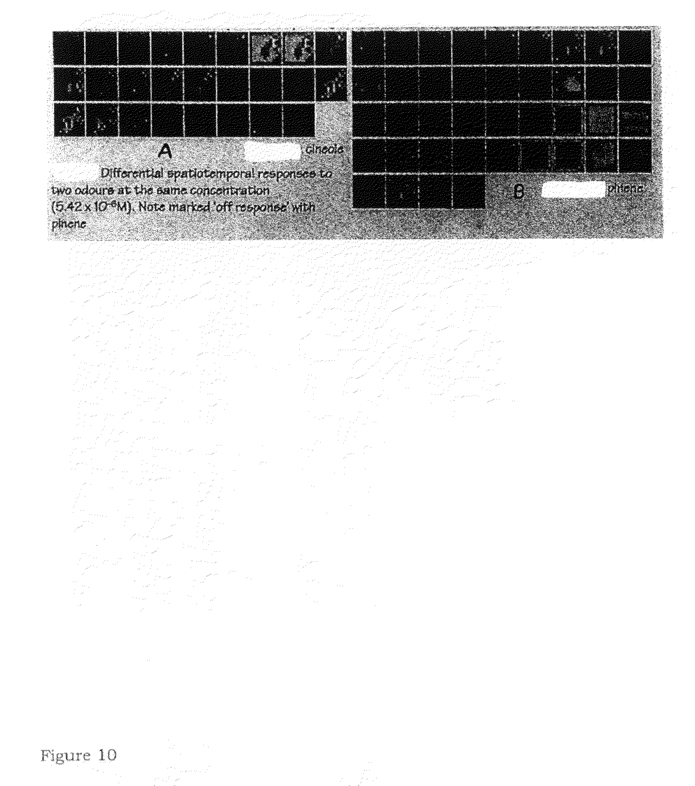

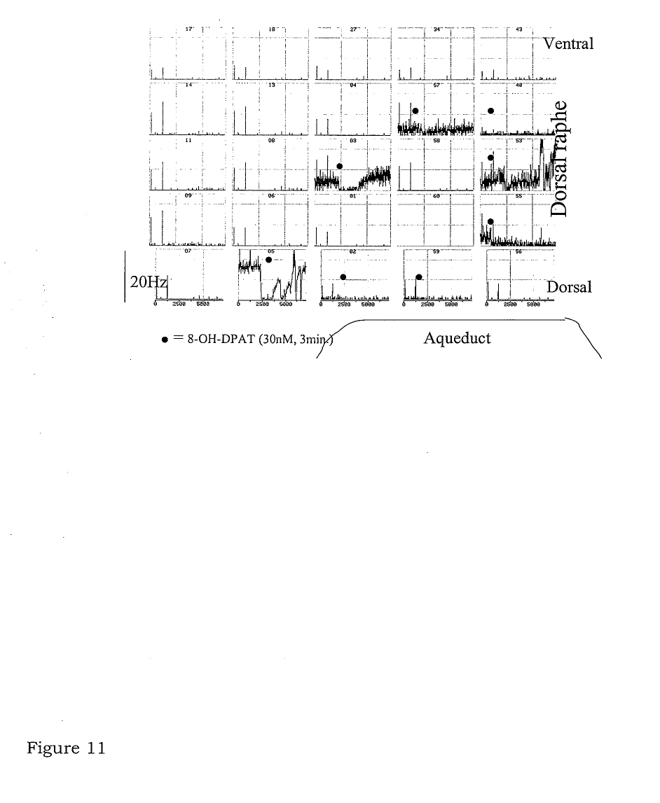

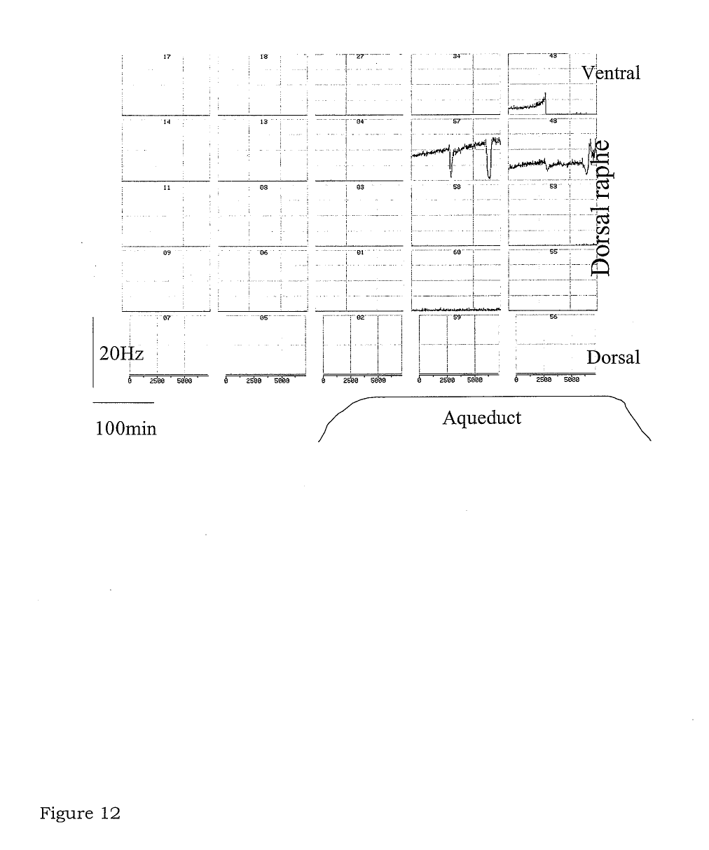

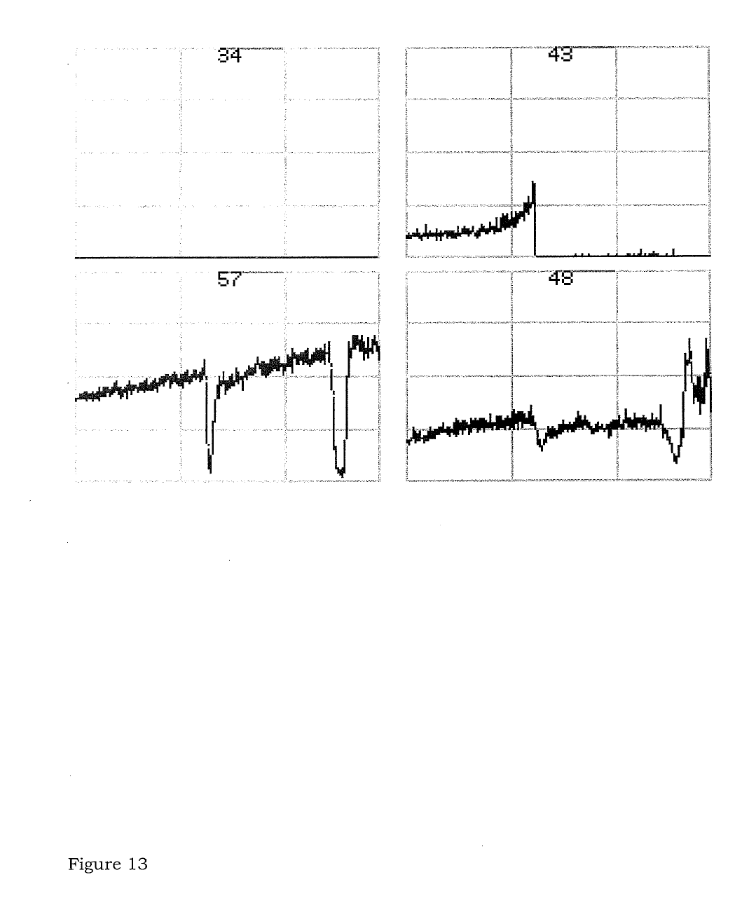

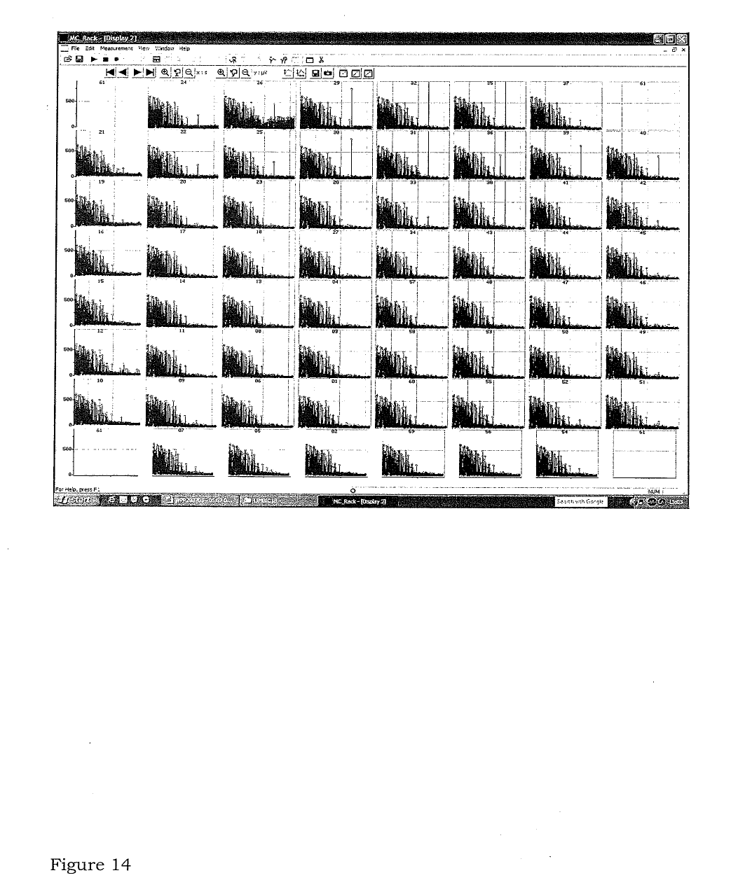

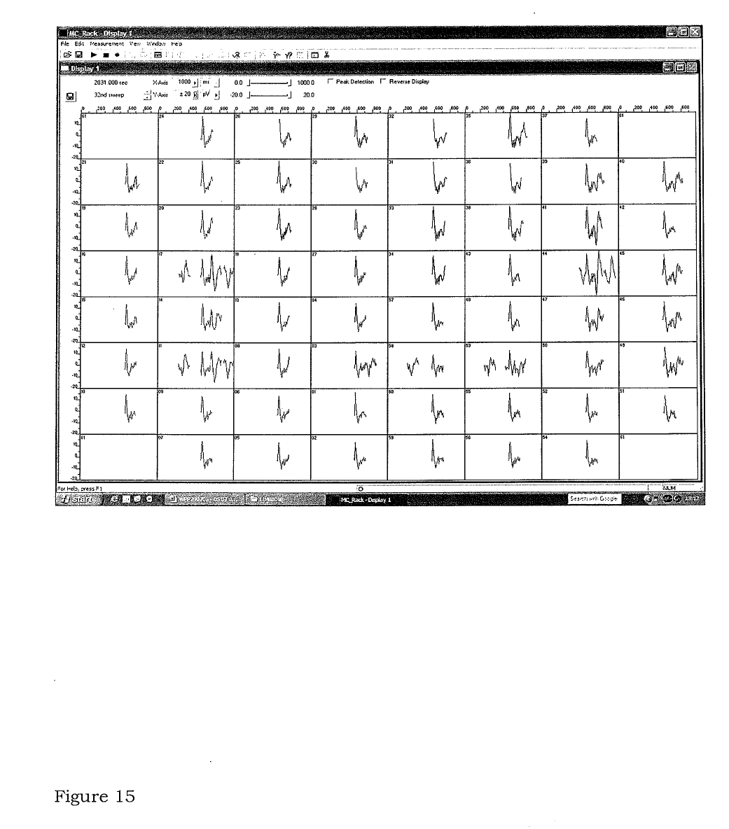

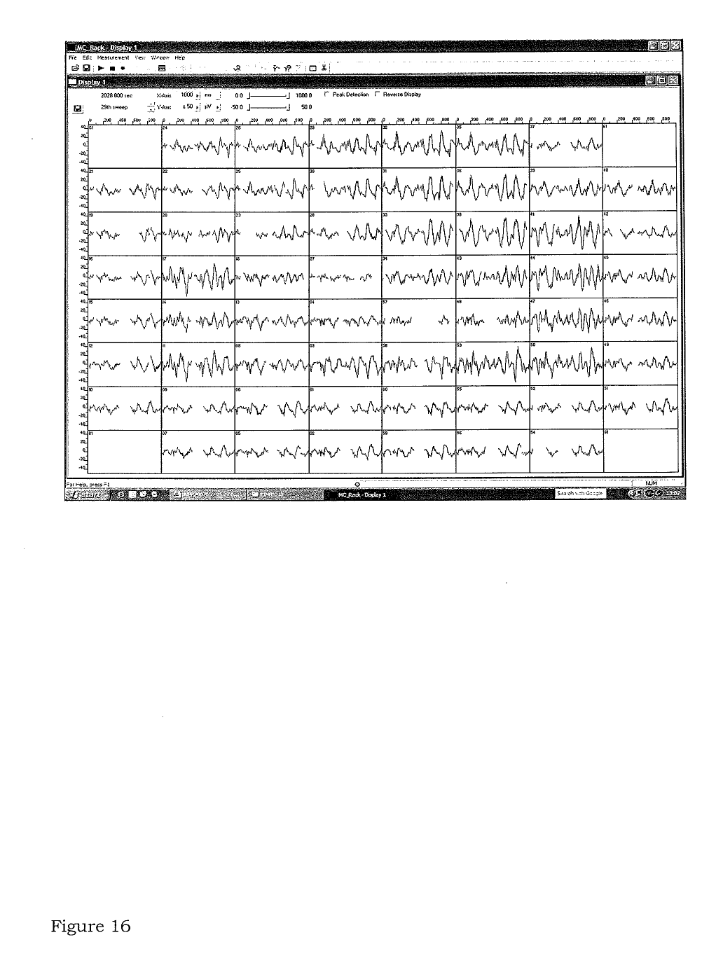

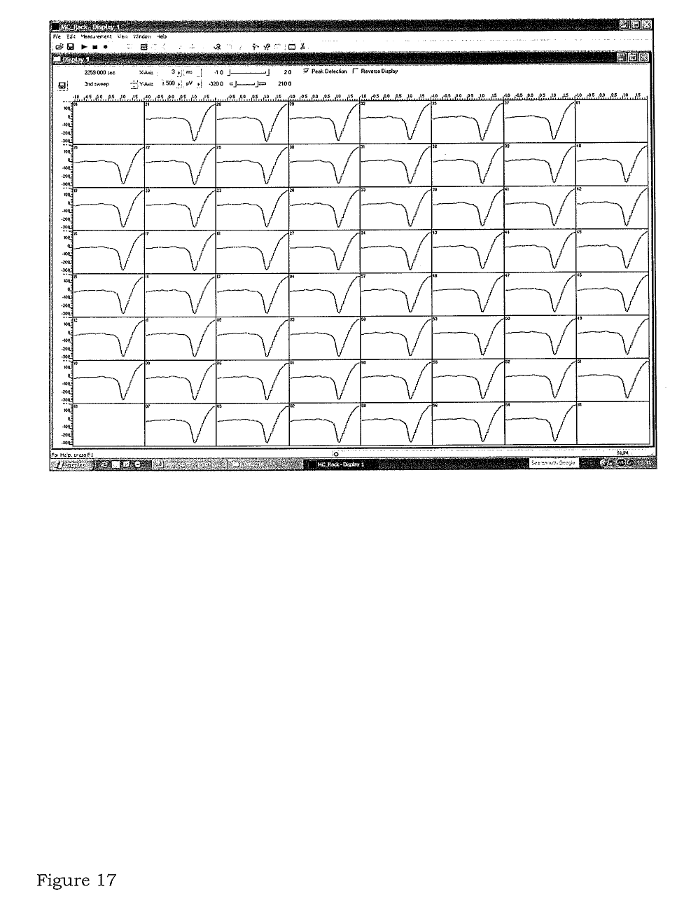

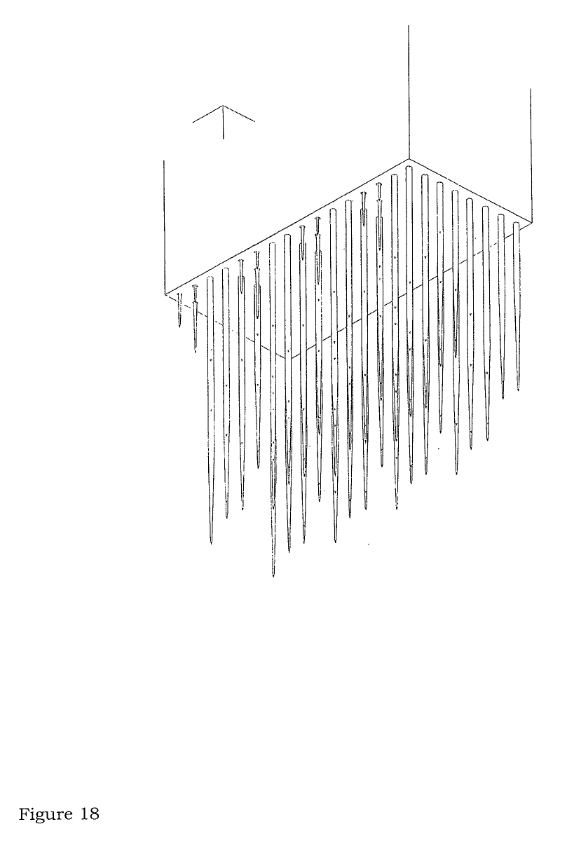

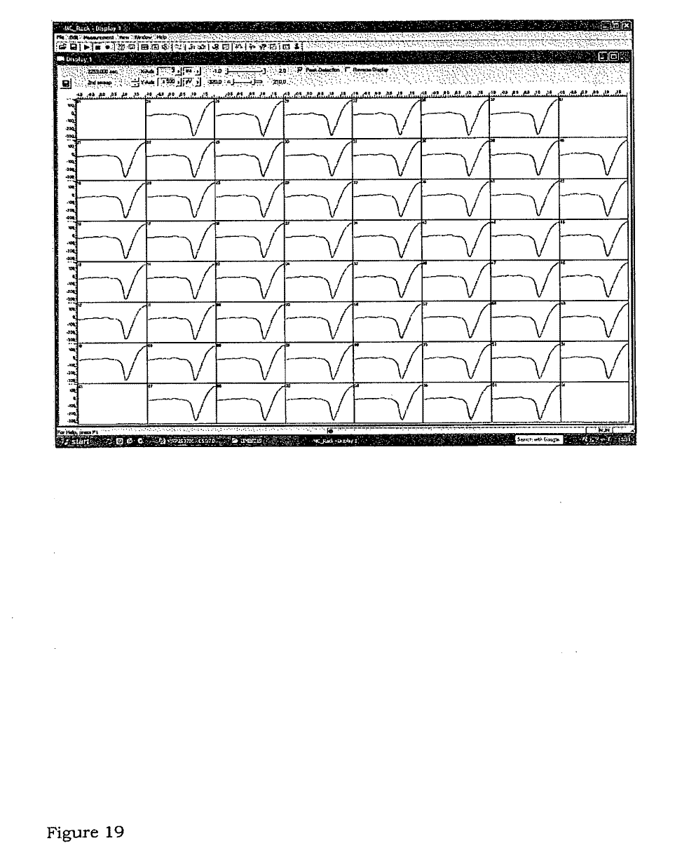

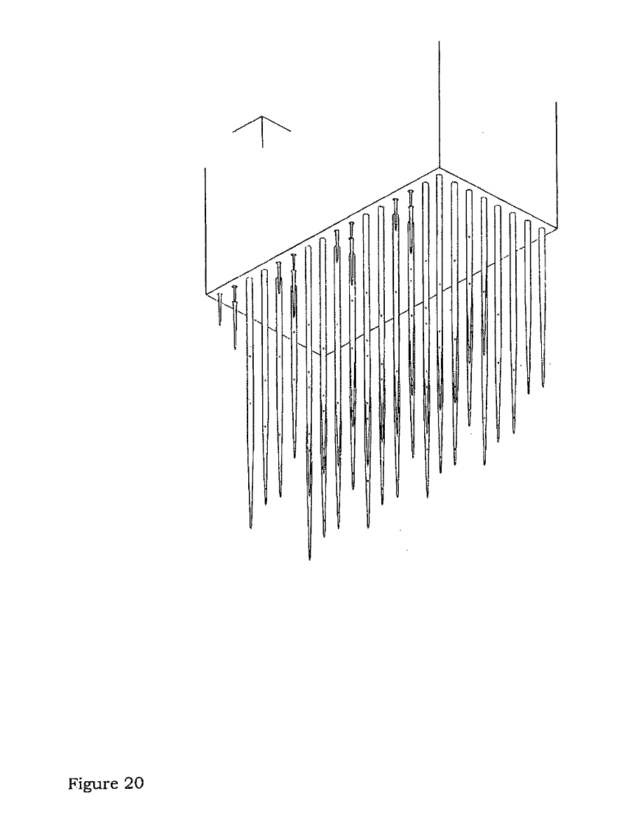

The most commonly used material for the individual conductive elements is tungsten. Thus has the advantage of being easily ‘micromachined’ by electrolytic means and is very rigid. Stainless steel is better for implantable devices, but is more difficult to work with. Impedance values in the range of 100-800 KOhm (e.g., 300 KOhm) are optimal for recording local field potentials (LFPs) and unit activity simultaneously with good signal/noise ratios. Square packing regimes are optimal for conducting various data analyses based on cytoarchitectonics (e.g. current source density analysis), whereas hexagonal packing has a small advantage as far as number of channels per unit volume of tissue (with the same mesh size of spacer) Spacer material is now available to allow any number of channels to be built up according to either packing regime.

The MEA of the invention can be made so that they are compatible with all major data acquisition systems and with all connector types, for example they will fit conventional “Michigan Probe” holders and all other carriers.

FIG. 2 shows an outline of the MEA data recording system of the invention.

FIG. 3 shows data obtained using known modulators of smooth muscle activity. The effect of known modulators of spontaneous muscle contraction of bladder muscle recorded using an MEA system (a 60 channel NeuroZond™ MEA of the invention was used). The trace shows the number of contractions in five minutes plotted against time of the experiment in minutes.

Application of acetylcholine (Ach, 100 μM) caused an increase in activity. Following recovery, the KCNQ2/3 activator retigabine (10 μM) inhibited phasic contraction. Increasing the perfusion solution temperature from 35° C. to 37° C. increased the amount of activity. Finally the L-type calcium channel antagonist nifedipine was added. This produced the expected block of all phasic activity.

FIG. 4 shows the effects on neuronal activity in smooth muscle (a 60-channel NeuroZond MEA of the invention was used). A. Initially the preparation was bathed in 12.5 μM nifedipine and this was changed to 50 μM nifedipine for the period from 1500 to 2000s. This led to an initial excitation followed by almost complete block of the activity. Although the traces are only ratemeter recordings it can still be seen that the firing patterns at this crude level of analysis are not the same on adjacent channels. B. Channel 22 is shown expanded.

FIG. 5 shows a photograph of a complete MEA system.

FIG. 6 shows a photograph of the MEA in use.

FIG. 7 shows that using an MEA system (a 60-channel NeuroZond MEA of the invention was used); the signal to noise ratio obtained is excellent, even from small cells within nuclear structures in vitro front rat ventromedial hypothalamus. Conventional slicing techniques were used, and the slices were perfused at approximately 4 mls/min. The cells in these structures produce small ‘closed’ electric fields because of their stellate geometry, being able to record from populations of these cells is a major advantage of the invention

FIG. 8 shows the activity of different neuronal types at each site and across the array in this case within rat prelimbic frontal cortex. This shows that spontaneous activity of both inhibitory interneurones (the smaller signals crossing the thresholds) and major projection cells (pyramidal cells) can be recorded simultaneously under normal physiological conditions (in conventional ACSF medium at 35° C.).

FIG. 9 shows a case study in which acute in vivo recordings were made in the olfactory bulb. Multi-electrode array recordings from olfactory bulb mitral cells (5×5 array). 1. Spontaneous population burst. 2. Control, no odour. 3. Arnyl acetate. 4. N-butanol. 5. Cineole. 6. Pinene. 7. DL-Camphor. 8. Linalool (max response). 9. Linalool “off response”. All at a concentration of 2.7×10⁻⁵ M.

The results are shown as peak odour response neuronal activity maps in olfactory bulb. These records were from male Wistar rats (400 g) anaesthetized with urethane and secured in a stereotaxic unit). The NeuroZonds MEAs of the invention penetrated the left olfactory bulbs horizontally (after left enucleation). The odours were presented in the animals breathing air under the control of the normal breathing cycle (detected by thermistor probe in mask), diluted from stock in pure nitrogen. The concentrations of odourants were determined according to the relative saturation vapour pressures of the odourants at particular temperatures via a custom-built ‘olfactometer’.

FIG. 10 shows that different spatiotemporal dynamics can be detected during acute in vivo recording in olfactory bulb for two odours (a) cineone, and (b) pinene (note marked “off response”), at the same concentration (5.42×10⁻⁶ M).

FIG. 11 shows an in vitro assay for an anti-obesity compound, the rate meter output of “glucose responsive” (GR) and “glucose sensitive” (GS) cells in rat ventromedial hypothalamus (VMH grey region, circled) on challenge with 3 mM glucose in the tissue bathing solution. GR cells show a drop in spontaneous firing when challenged with 3 mM glucose, and GS cells show the converse. Compounds mimicking the effects of 3 mM glucose on GR cells may be of use as anti-obesity drugs, as GR cells form an important part of the “satiety circuit”.

FIG. 12 shows a graph of time (sec) against firing (% from 5000-6000 sec) for data obtained in an in vitro assay for anti-obesity shown as the rate meter output of “glucose responsive” neurones in rat VMH, challenge with 3 mM glucose was followed by introduction of drug PYM50057 at 1 μM (▪), 3 μM () and 10 μM (▴).

FIG. 13 shows the effect of 8-OH-DPAT () 30 nM, 3 min on neuronal firing: rat dorsal raphe of the midbrain/MEA. These cells contain serotonin, and are of particular importance in the control of mood and sleep patterns.

FIG. 14 shows the effect of retigabine and XE-991 on neuronal firing: rat dorsal raphe/MEA. Retigabine and XE-991 act on a particular family of potassium channels (KCNQ).

FIG. 15 shows recordings for channels 34, 43, 48 and 57 (of FIG. 14) that demonstrate recovery of retigabine inhibition by XE-991 (note heterogeneity).

FIG. 16 shows peak-to-peak (p-p) amplitude frequency: an example of the effects of a test compound (10 μM) targeted at the beta-subunits of N-type calcium channels on epileptic activity in a horizontal slice of lateral entorhinal cortex. The first 2000 seconds represent a control period with re-circulating zero-magnesium ACSF (artificial cerebrospinal fluid). At 4000 sec., this is switched over to an ACSF reservoir that contains the compound under test. The plots display the occurrence of ‘epileptic features’ in the LFP records (small circles), and their p-p amplitude (on the Y-axis) against time. If there is more than one of these events in a 10 second bin, the range is joined by a vertical line.

FIG. 17 shows the first type of epilepsy-associated activity in the LFP records, namely, ‘interictal spikes’ with various degrees of synchronization across the array.

FIG. 18 displays an example of an array-wide synchronized full-blown ictal (or electrographic) seizure.

FIG. 19 illustrates an example of synchronized unit firing recorded simultaneously with the LFP data stream. These large amplitude unit firings are associated with the beginning of ictal seizures. These spikes are actually the first discharge within a high-frequency burst.

FIG. 20 shows a chronic in vivo cranial implant with electrodes of variable length. This type of implant was produced using stainless steel mesh (100 mesh) spacer material sheets. The individual conductive elements were arranged on repeating length sequences in 500 micron steps (500 microns to 8 mm) throughout the array. (NB the scale bars are 1 mm). The main targets for such implants are various parts of the cortex or spinal cord of larger vertebrates such as primates, and they are implanted after removal of the dura, and enzymatic softening of the pia under sterotaxic/X-ray control during general anaesthesia. Such devices are not only of use for research purposes, but represent a step forward in true ‘neural prostheses’, especially as they can pole many different neural layers simultaneously.

### EXAMPLES

### MEA Methods

### 1. Neuronal Tissue (Brain Slice)

Wistar rats or mice obtained from Charles River were sacrificed by cervical dislocation. The brain was rapidly removed and a block of tissue containing the relevant area was prepared. Brain tissue was glued onto a glass slide using cyanoacryalate glue (Permabond C2). The block was supported by a similar sized block of agar (3% in NaCl, 126 mM) glued immediately behind the brain block. Coronal slices (300 μm thick) of the relevant brain slice were cut from the brain using a Vibratome™ (Oxford Instruments, supplied by Intracel, Unit 4, Station Road, Shepreth, Royston. Herts. UK). The slices were transferred to artificial cerebrospinal fluid (ACSF) which contained (in mM): NaCl 126, KCl 5, NaH₂PO₄ 1.24, CaCl₂ 2.4, MgCl₂ 1.3, NaHCO₃ 26, glucose 10, at pH 7.4, continuously bubbled with ‘carbogen’ (95% O₂/5% CO₂) and maintained at room temperature.

For recording, individual slices were transferred to a specialised multi-electrode array (MEA) recording chamber/electronic interface combination where they were perfused with ACSF at 33.2-35.4° C. (typically 35° C.) with glucose (10 mM), flowing at 4-4.5 ml/min. The multi-electrode array system consisted of 60 electrodes in a regular 8×8 array (four corner channels are common ground, thus allowing recording from 60 electrodes).

The MEA forms part of a ‘wandering probe’ (NeuroZond™: designed and constructed ‘in-house’ as described herein) that can be positioned, using a dorsal approach under microscopic control (video microscope: OCU-CAM, PCE Power Control) in 3 dimensions relative to the slice. This technique allowed consistency of electrode placement between preparations using cytoarchitectural landmarks in the slices. The individual electrodes in the NeuroZond MEAs are made from tungsten fibres, electrolytically etched to a fine tip, insulated with three layers of Epoxylite, and ‘micromachined’ to give specified tip geometries/impedence values allowing easy, atraumatic slice penetration, (impedence values of 100-800 KΩ). Interelectrode spacing was 250 μM.

Extracelular action potentials were recorded from either isolated neurones, or several neurones per electrode. Due to the geometry and dimensions of the arrays, it was possible to sample representative neural populations or ensembles, not only from the target structure but also from other nuclei/layers situated in close proximity. The individual electrodes in the various arrays are continuously connected via a cluster of 8-channel headstage amplifiers with a fixed ×10 gain (MPA-8; Multi Channel Systems, MCS GmbH, Aspenhaustrasse 21, 72770 Reutingen, Germany; supplied by Scientifica, Herts). These pre-conditioned, wide-band signals are then fed to a 64-channel programmable amplifier, via junction and splitter boxes, and from there to one half of a 128-channel data acquisition board mounted in a high power, twin-CPU laboratory computer. Data are acquired and displayed by a ‘virtual instrument’ software package, MCRack (MCS; Multi Channel Rack V3.5.1), and the following recording parameters were employed throughout: total gain of 5000×, band pass of 300 Hz to 6 KHz (for unit activity), or 0.1 Hz-100, 150, or 300 Hz (for local field potentials: LFPs), 25 K sampling rate (A/D conversion) per channel, A/D voltage range of −4096 to +4095 mV.

All spike waveforms or LFP ‘events’ that crossed a pre-set voltage threshold (−12 and −8 pV respectively) were stored as both ‘time-stamps’ (i.e. ‘time of occurrence’), and as ‘spike cut-outs’ (i.e. digitised waveform prior to, and after threshold crossing). All experimental data were recorded directly on to hard disk, and backed up onto various means of data archiving.

Ratemeter histograms were constructed for each of the data channels. In this way it is possible to build up a topographical representation of spontaneous activity. Test substance was applied via the perfusing system (either by gravity feed ‘to waste’, or recirculated at concentrations depending on the tissue/compound, but usually in the range of from 0.1 μM to 100 μM) and the effects on spontaneous activity determined, together with measurement of recovery time following removal of test substance.

### 2. Smooth Muscle (Bladder)

Wistar rats or mice from Charles River were sacrificed by cervical dislocation. The bladder was removed, opened and pinned out as a flat sheet on a Sylgard dish. For overactive bladder studies the urothelium was removed.

The bladder sheet was transferred to a recording chamber where it was perfused with artificial cerebrospinal fluid (ACSF) which contains (in mM): NaCl 126, KCl 5, NaH₂PO₄ 1.24, CaCl₂ 2.4, MgCl₂ 1.3, NaHCO₃ 26, glucose 10, at pH 7.4, continuously bubbled with ‘carbogen’ (95% O₂/5% CO₂) and maintained at 35° C., flowing at 4.5 ml/min.

The multi-electrode array system consisted of 60 electrodes in a regular 8×8 array (four corner electrodes are common ground, thus allowing recording from 60 electrodes).

The MEA forms part of a ‘wandering probe’ (NeuroZond: designed and constructed ‘in-house’ as described herein) that can be positioned, using a dorsal approach under microscopic control (video microscope: OCU-CAM, PCE Power Control) in 3 dimensions relative to the bladder sheet. This technique allows consistency of electrode placement between preparations using cytoarchitectural landmarks in the sheet. The individual electrodes in the NeuroZond MEAs are made from tungsten fibres, electrolytically etched to a fine tip, insulated with three layers of Epoxylite, and ‘micromachined’ to give specified tip geometries/impedence values allowing easy, atraumatic tissue penetration, (impedence values as previously described). Interelectrode spacing is 250 μM.

Extracellular muscle action potentials are recorded from either isolated detrusor muscle cells or several cells per electrode. The individual electrodes in the various arrays are continuously connected via a cluster of 8-channel headstage amplifiers with a fixed x 10 gain (MPA-8; Multi Channel Systems, MCS GmbH, Aspenhaustrasse 21, 72770 Reutingen, Germany; supplied by Scientifica, Herts. UK). These pre-conditioned, wide-band signals were then fed to a 64-channel programmable amplifier, via junction and splitter boxes, and from there to one half of a 128-channel data acquisition board mounted in a high power, twin-CPU laboratory computer. Data were acquired and displayed by a ‘virtual instrument’ software package, MCRack (MCS; Multi Channel Rack V2.2.2.1), and the following recording parameters were employed throughout: total gain of 5000×, band pass of 100 Hz to 1 KHz, 25 K sampling rate (A/D conversion) per channel, A/D voltage range of −4096 to +4095 mV.

All spike waveforms that crossed a pre-set voltage threshold (−15 μV) were stored as both ‘time-stamps’ (i.e. ‘time of occurrence’), and as ‘spike cut-outs’ (i.e. digitised waveform prior to, and after threshold crossing). All experimental data were recorded directly on to hard disk, and backed up onto compact disc.

Ratemeter histograms were constructed for each of the data channels. In this way it was possible to build up a topographical representation of spontaneous activity. Test substance was applied via the perfusing system (as described for neural tissue) and the effects on spontaneous activity determined, together with measurement of recovery time following removal of test substance.

Whilst recording high frequency electrical activity from the bladder sheet the MEA can also be used to record low frequency signals as a result of muscle contraction. A video camera placed beneath the sheet can also be used to videotrack the spontaneous muscle movement.

### 3. MEA—Data Using Known Modulators Of Smooth Muscle Activity.

The effect of known modulators of spontaneous muscle contraction of bladder muscle recorded using the MEA system described above. In FIG. 3, the trace shows the number of contractions in five minutes plotted against time of the experiment in minutes.

Application of acetylcholine (Ach, 100 μM) caused an increase in activity. Following recovery, the KCNQ2/3 activator retigabine (10 μM) inhibited phasic contraction. Increasing the perfusion solution temperature from 35 to 37° C. increased the amount of activity. Finally, the L-type calcium channel antagonist nifedipine was added. This produced the expected block of all phasic activity.

### 4. MEA—Effects on Neuronal Activity in Smooth Muscle

FIG. 4A shows the recording of electrical activity across the entire MEA. Initially the preparation was bathed in 12.5 uM nifedipine and this was changed to 50 uM nifedipine for the period from 1500 to 2000s. This led to an initial excitation followed by almost complete block of the activity. Although the traces are only ratemeter recordings it can still be seen that the firing patterns at this crude level of analysis are not the same on adjacent channels. One channel, channel 22, is shown expanded in FIG. 4B.

### 5. Analysis Algorithms for Multi-Electrode Array (MEA) Data

The key problem in the meaningful analysis of the prodigious quantities of data generated from such experiments lies in the fact that information of use to the experimenter is embedded in the complex spatiotemporal dynamics expressed both within and between large populations or networks of neurones. Conventional techniques for the analysis and representation of extracellularly recorded neuronal discharges, or ‘spikes’ (such as the production of ratemeter output, which graphically represents the changes in firing frequency on a single channel plotted against time) are incapable of extracting pertinent information from such data sets.

Some newer techniques have been developed to analyse data derived from multi-electrode recordings, such as ‘gravitational clustering’, and its developments, and the construction of joint-peri-stimulus time histograms Ooint-PSTH) and ‘population vectors’. However, these techniques are mostly based around the multiple pairwise analysis of the relationships between the individual cells within the population sampled, usually time-locked to a specific event (sensory or electrical stimulus, or motor output). In such a form, these methods are not ideally suited to the massively parallel and extensive electrophysiological recordings that are inherent to the MEA-based assays.

Data analysis techniques are required that inherently incorporate spatial dimensions in their structure. By implementing such techniques ‘spatiotemporal neural activity signatures’ of epilepsy in each of the regions studied in vivo will be produced.

The data sets obtained from NeuroZond MEA recordings are initially processed using two distinct strategies, the results of which are then fed into a final common path, based upon an ‘Artificial Neural Network’ (ANN) in order to yield a ‘pathology index’, compared to the ‘native’ condition.

The spike signals derived from each of the NeuroZond MEA electrode channels are processed through two different integration protocols.

The first protocol involves the clipping of one polarity of the signal (‘½ wave rectification’) in order to prevent algebraic addition or cancellation of the potentials downstream. This ‘rectified’ signal is then applied to a ‘leaky integrator’ having a time constant (τ) of 10-15 ms (accuracy better than 2%). This procedure will provide the experimenter with DC analogue voltages that represent the ‘activity envelopes’ or ‘power’ (analagous to RMS) of the overall multi-unit activity (MUA) on each of the channels in the array. Such procedures can be implemented in both hardware and software.

In order to obtain similar output related to the firing of individual neurones (often 3-4 different cells or ‘units’ can be recorded by any single electrode in the array), spike waveform discrimination/separation (also known as ‘spike sorting’ protocols are implemented. A method of spike discrimination that has been found to be particularly effective is ‘continuous time principal components sorting’. The good performance of this technique can be understood when considering the fact that the first principal component (PC) equals the normalized average of the action potential waveforms, and the second PC measures differences in waveform shape, and often appears similar to the temporal derivative of the first component. These features are very nearly equivalent to the theoretically optimal pair of features for classifying detected spikes.

Principal components analysis can be performed in real time utilizing digital signal processing chips (DSPs). However, during the early stages of this programme of work, an off-line version of PC analysis of spike shape can be implemented utilizing the ‘Spike Tools’ component of the MEA-Tools package (ver. 2.62) within the MATLAB (ver. 6.5/7.0) environment¹⁷⁵ (The MathWorks, USA). ‘Parsing’ of the data files into a MATLAB ‘datastream object’ creates a record in the MATLAB workspace containing all file header and data record information concerning spike times (‘time stamps’), associated channels, and the calculation of the PCs for each channel. Clusters of the component scores are then identified and delimited. (‘cluster cutting’) in pair-wise two-dimensional projections of ‘feature space’ (this can be increased to three dimensions). The procedure is extended by utilities to review individual spikes and groups of spikes, identify meaningful groupings, and tag them for assignment to a particular cluster.

Development of the MEA-Tools open source software is used to improve these spike sorting protocols in two stages. Firstly, the ‘cluster cutting’ procedures instituted in multidimensional space can be automated using advanced classification algorithms, including K-means and ‘valley seeking’. Secondly, more extensive programming in the MATLAB environment and use of the MATLAB Wavelet Toolbox produces spike sorting tools based around ‘wavelet packet decomposition’ (WPD). This method is more effective than PC methods in both separating small spikes from background noise, and in resolving temporally overlapping spikes.

Once spike sorting procedures are completed, the ‘spike train’ files containing the times of occurrence of individual spikes (i.e. their time stamps) can be directed to the software-based integrator mentioned previously, and henceforward treated as per the integrated MUA signals.

The following specialized analyses in the time domain (i.e. time series analyses) can be executed utilizing a combination of functions within the NeuroExplorer (Nex) package, and the Signal Processing and Statistics Toolboxes of MATLAB. These procedures represent an implementation and extension of techniques initially developed by Professor Guenter Gross and his colleagues at the Center for Network Neuroscience, University of North Texas, Denton, USA.

Initially, eighteen ‘unit activity variables’ will be determined for one minute epochs for each channel throughout an experimental episode (the length of which is determined through pilot experiments and is in the range from 60-300 minutes). These experimental episodes will be comprised of: (i) the native condition, (ii) the presence of electrophysiological correlates of interictal activity (and polyspiking), and (iii) the presence of electrophysiological correlates of full blown ictal seizures (status epilepticus).

The first ten variables will be determined from the spike ‘time stamps: (1) Spike Rate (SR: spm), (2) Change in SR (%), (3) Number of Spikes in Bursts (NSIB), (4) Number of Spikes Not in Bursts (NSNIB), (5) Spikes in Bursts (SIB: %), (6) Spikes Not in Bursts (SNIB: %), (7) Spike Mean Frequency (SMF: Hz), (8) Spike Peak Frequency (SPF: Hz), (9) Modal Interspike Interval in Bursts (MIIB: ms), and (10) Modal Interspike Interval Not in Bursts (MIINIB: ms). The Burst Detector/Analysis component of Nex (including Poisson surprise S) can be employed.

The next eight variables are derived from the output of the integration procedures described above, but will also use the Nex Burst Detector: (11) Burst Rate (BR: bpm), (12) Change in Burst Rate (%), (13) Burst Duration (BD: sec.), (14) Burst Power (MUA) (BP_{MUA}: standardized ‘area-under-the-curve), (15) Burst Power (UNIT) (BP_{UNIT}: standardized ‘area-under-the-curve), (16) Burst Amplitude (BA: standardized integrated units), (17) Burst Period (BPr.: sec.), and (18) Interburst Interval (IBI: sec.).

From the above ‘unit activity variables’, the degree of temporal regularity and network synchronization present during the various experimental episodes are computed. This can be achieved by producing two different coefficients of variation (CVs) for each of the activity variables described above. All calculations are based on the contents of the one minute recording epochs. These values are used to obtain experimental episode means for each channel with CVs for experimental episodes, and ‘minute means’ for each minute across the whole network (i.e. all sampled channels). The experimental episode CVS (CV_{TIME}) for each channel represents a measure of temporal pattern fluctuation for that channel across the episode. Averaged across the network, CV_{TIME} reflects pattern regularity for the network, even if several patterns exist, and even if they are not synchronized. Conversely, the minute CVs (CV_{NETWORK}) represent channel coordination. Averaged across the experimental episode, CV_{NETWORK} reflects the degree of network synchronization, even if the pattern fluctuates in time.

In addition to those described above, a further experimental activity variable, that of Response Delay Time (RDT, or latency: sec.) can be obtained during experimental episodes during transitions from the native condition, to the presence of the various electrophysiological correlates of epilepsy (detected mostly by the appearance of interictal spiking, polyspikes, and/or ictal seizures in the LFP records as described above). The RDT will be determined using modifications of the cumulative sum statistic (CUSUM) in Nex.

In order to produce spatiotemporal neural activity signatures in the time domain for the experimental episodes (i) native condition, and (ii) presence of electrophysiological correlates of epilepsy, the squared deviations of each of the unit activity variables are calculated and summed across the array to yield Coefficients of Identification (CIs), thus: CI_{(NATIVE OR EPILEPSY)}=√Σ(mean_{variable}/SD_{variable})².

Thus, an ‘index of efficacy’ of any drug being tested against epileptiform activity can be obtained in this domain by calculating the ci_{(drug)} in an identical manner, and observing where its value lies on the ci₍ₙₐₜᵢᵥₑ₎−ci_{(epilepsy)} continuum. If these operations are repeated using the minute means for each of the variables across all of the channels, then a good approximation of the time course of the drug's effects will be obtained.

A final important measure of the behaviour of the neural networks sampled (in the time domain) during the different experimental episodes will be an estimation of the presence and magnitude of oscillatory activity. This is because it is possible for both a high degree of pattern regularity, reflected by CV_{TIME}, and synchronization, reflected by CV_{NETWORK}, to be present across the array, without the activity of the sampled network being oscillatory. Thus, this measure represents an additional useful characterizing network statistic.

Firstly, autocorrelation functions will be computed for each discriminated unit on each channel (timespan −30 to 30 s, bin size 50 ms) separately for the different experimental episodes, and averaged across the array. Subsequently, the Gabor function (a damped harmonic oscillation) will be fitted to the resultant histograms (with the exponent λ set to 2). This operation will yield the following descriptive parameters: (i) frequency, ν, of the harmonic oscillation, (ii) amplitude, A, (iii) decay of the Gaussian curve, σ, and (iv) offset above the abscissa, O. Finally, the coefficient ‘decay amplitude’ can be derived from these parameters as a measure of the presence and degree of oscillation. A coefficient greater than two is considered to indicate oscillatory neural activity, which is of particular importance (see below).

The final set of analyses that can be conducted in the time domain are aimed at extracting any signature sequences, structures and patterns in the spike trains recorded across the arrays that represent ‘fingerprints’ of the native state, or the neural network correlates of epilepsy. This can be achieved by conducting kernel, linear, and non-linear ‘canonical correlation analyses’. It should be noted that these techniques require the construction and running of an Artificial Neural Network (ANN; also see section below concerning the ‘final common path’), and will therefore be implemented using the Neural Network Toolbox of MATLAB.

In contrast to the above, the second major strategy involves analysis of the array-derived spike data ‘in the frequency domain’. Generally speaking, these procedures are theoretically and computationally more sophisticated than those executed ‘in the time domain’.

Three multivariate techniques to analyse the multichannel (neural ensemble) data are proposed, all of which are centred on the idea of extracting meaningful statistics from the ‘spatiotemporal neural population activity maps’.

Linear Discriminant Analysis (LDA) can be used as a ‘classifier’ of the spatiotemporal neural population activity maps associated with each of the experimental episodes described above.

Independent Coding Analysis (ICA) can be used to identify groupings of neurones recorded by the arrays that may be ‘emergent’ during the different experimental episodes due to higher-order correlations that are not simply produced by the neurones' covariance.

Finally, Principal Components Analysis (PCA) can be used to look for similar neural ensemble correlations in multidimensional space. However, it should be noted that the ‘population vectors’ derived using this technique tend to be broadly distributed over the sampled neural population, and thus PCA is unable to identify independent groupings of neurones that may share common sources of input. However, PCA may yield significant data under the highly phasic population activity patterns that are observed in the olfactory-limbic axis when expressing neural correlates of epilepsy.

Another frequency domain multivariate technique, that of Partial Directed Coherence (PDC) enables causal relationships (i.e. directionality of information flow/propagation of epileptic activity) between these structures to be determined. Any perturbation of basal/native ‘directionality’ caused by the induced in vivo epileptiform activity can be quantified, as, therefore, can the ‘normalising power’ of any putative AED-candidate compound.

The detection and quantification of the various LFP expressions of epileptic activity (interictal spikes, polyspikes, and tonic/clonic ictal seizures), and the filtering of artefacts can be carried out in parallel with the above unit analyses. This can be accomplished by the utilisation of pattern recognition and extraction techniques similar to those developed by Ayala and colleagues. In addition, LFP signal power spectrum analysis can be conducted continuously for all channels, producing data for the square of the root mean-squared voltage (Vᵣₘₛ)².

Array-wide ‘correlation analysis’ can also be conducted for all conditions. During ictal recordings, when signals are dynamically varying, the Pearson correlation coefficient will be used, whereas during interictal/polyspike periods, when signals approach steady state, it becomes possible to estimate reliably the ‘magnitude squared coherence’ (MSC), which has the advantage over simple Pearson correlation in that it provides information concerning specific frequency bands.

MSC spectra can be broken down into the following standard frequency bands: ‘delta’ (>0 to <4 Hz), ‘theta’ (4 to <8 Hz), ‘alpha’ (8 to 13 Hz), ‘beta’ (>13 to <30 Hz), and ‘gamma’ (30 to 80 Hz). Magnitude squared coherence (MSC) will provide a linear measure of the relationships between the LFP ‘epilepsy signatures’ recorded from the different channels of the arrays, with the ability to specify the contributions of the different signal frequencies just mentioned, that is, MSCs can be interpreted as correlation coefficients with a frequency index. It should be noted that prior use of ‘coherence’ in epilepsy research has been used to study propagation delay during seizure, and the spatial and temporal structure of intracranial EEGs.

The last form of LFP analysis that can be conducted is autoregression. This is used to gain information concerning the dynamic changes in ‘propagated’ versus ‘independent’ afterdischarges and the relative contributions of the different structures in the olfactory-limbic axis to the focal epilepsies studied, and will also examine how these features are affected by the various putative AED compounds tested.

The final common path for the entire analysis procedure described above is to feed the results of both the time and frequency domain procedures applied to the unit data, together with the results of the various LFP analyses into an ANN for feature extraction and pattern recognition of the different experimental states, and consequently obtain comparative values for a definitive index of efficacy of the various compounds tested.

1. A multi-electrode array comprising a plurality of electrodes, the electrodes being spaced apart from each other by spacer means, the electrodes being secured to the spacer means, the spacer means being encapsulated within a housing.

2. A multi-electrode array according to claim 1 wherein the spacer means and housing are unitary.

3. A multi-electrode array according to claim 1 wherein the electrodes have a diameter in the range of from about 30 μm to about 200 μm, preferably from about 40 μm to about 150 μm, e.g. 125 μm.

4. A multi-electrode array according to claim 1 wherein the electrodes comprise tungsten, stainless steel, platinum, platinum/iridium, carbon fibres, conductive nanotubes, carbon nanotubes, an Elgiloy® alloy, or a conductive polymer.

5. A multi-electrode array according to claim 1 wherein the electrodes are metal electrodes.

6. A multi-electrode array according to claim 5 wherein the electrodes have shape memory.

7. A multi-electrode array according to claim 6 wherein the electrodes are formed from stainless spring steel.

8. A multi-electrode array according to claim 1 wherein the electrodes are wires.

9. A multi-electrode array according to claim 1 wherein at least one of the electrodes is hollow.

10. A multi-electrode array according to claim 1 wherein the spacer means comprises a plurality of electron microscopy grids, a plurality of fine mesh patches, or a block containing channels.

11. A multi-electrode array according to claim 1 wherein the electrodes are spaced in a regular pattern or a regular repeating pattern.

12. A multi-electrode array according to claim 1 wherein the electrodes are spaced equidistant from each other.

13. A multi-electrode array according to claim 1 wherein one or both of the housing and spacer means are formed from a mouldable material.

14. A multi-electrode array according to claim 13 wherein the mouldable material is an acrylic polymer or a thermoplastic material.

15. A multi-electrode array according to claim 1 wherein the electrodes are releasably secured to the spacer means.

16. A multi-electrode array according to claim 1 wherein the electrodes tips are in a non-planar configuration, or are adjustable to a non-planar configuration.

17. A multi-electrode array wherein each electrode comprises a connector for connection to a driver.

18. A multi-electrode array wherein each electrode comprises a push fit connector.

19. A multi-electrode array according to claim 1 wherein electrodes are provided with an electrical insulating layer or jacket.

20. A multi-electrode array according to claim 1 wherein the electrodes are dual function electrical signal and mechanical transduction detectors.

21. An implantable device comprising a multi-electrode array according to claim 1.

22. A method for monitoring the effect of a test substance on a biological tissue comprising:
(a) providing a biological tissue in a medium,
(b) contacting the electrode tips of a MEA, in particular according to claim 1, with the tissue sample,
(c) recording the electrical signal detected by one or more of the electrodes,
(d) optionally recording movement in the tissue by recording mechanical transduction of the one or more electrodes and/or using optical means,
(e) exposing the tissue to a test substance,
(f) recording the electrical signal detected by the one or more electrodes,
(g) optionally recording the movement in the tissue by recording mechanical transduction of the one or more electrodes and/or using optical means,
(h) comparing the results recorded in the absence and presence of the test substance.

23. A method according to claim 22 wherein the tissue is subjected to excitation prior to exposure to the test substance.

24. A method according to claim 22 wherein the tissue is selected from: smooth muscle, cardiac muscle, skeletal muscle, spinal cord tissue, brain tissue and secretory tissue.

25. A method according to claim 22 wherein each electrode is contacted with a single layer within the tissue.

26. A method according to claim 22 wherein the movement of the tissue is recorded using optical means.

27. A method according to claim 22 wherein a voltage sensitive dye or calcium sensitive dye is included in the medium.

28. A method according to claim 22 wherein the results recorded in the presence and absence of the test substance are compared using data analysis software.

29. A method according to claim 22 further comprising performing a patch clamp assay on a cell or cells within the tissue.

30. A method of manufacturing metal electrodes comprising:
(a) providing a plurality of metal wires spaced apart from each other in a substantially parallel manner and secured to a mandrel,
(b) forming an electrical connection between the wires,
(c) attaching the electrical connection to a first port of an AC electrical power supply,
(d) repeatedly dipping the metal wires into and out of an etch solution to a predetermined depth, a low voltage AC current being provided between the wires and a conductor within the etch solution, the conductor being attached to the second port of the AC electrical power supply,
(e) washing and drying the electrodes formed by electroetching the wires.

31. A method according to claim 30 wherein the metal wires are Tungsten wires.

32. A method according to claim 31 wherein the initial diameter of the Tungsten wires is in the range of from about 100 μm to about 150 μm.

33. A method according to claim 31 wherein the etch solution comprises Levick's solution.

34. A method according to claim 30 wherein the metal wires are stainless steel wires.

35. A method according to claim 34 wherein the etch solution comprises potassium cyanide or concentrated sulphuric acid.

36. A method according to claim 30 further comprising providing the electrodes with a layer of insulating material.

37. A method according to claim 36 wherein insulating material is selected from epoxylite resin, glass, fused quartz, polyimide, PARYLENE-C™, urethan, diamond, and FORMVAR™.

38. A method according to claim 30 wherein the insulating material is an epoxylite resin.

39. A method according to claim 30 further comprising de-insulating the electrode tips.

40. A method according to claim 30 further comprising testing electrical impedance of the electrodes.

41. A method according to claim 30 further comprising performing a second etch on each electrode to form the electrode tip to a desired dimension and impedance.

42. A method according to claim 30 further comprising testing impedance of the electrodes after said second etch.

43. A method according to claim 30 further comprising coating the electrode tip with gold.

44. A method according to claim 43 further comprising coating the gold-coated electrode tip with platinum black.

45. A method of manufacturing a set of metal electrodes for use in a microarray, the method comprising connecting precursor electrode wires to a common electrical conductor and electrochemically etching the electrode wires together in an etching bath to create a narrowed tip on each of said wires, whereby said method creates a substantially matched set of electrodes.

46. A matched set of electrodes, in particular produced by a method according to claim 45, whereby the electrodes have substantially matched electrical impedance.

47. A method of manufacturing a multi-electrode array comprising:
(a) providing a plurality of electrodes,
(b) spacing electrodes apart from each other by threading each electrode through a different gap or channel of a spacer means,
(c) securing the electrodes to the spacer means,
(d) encapsulating the spacer means within a housing.

48. A multi-electrode array (MEA) having a plurality of microelectrodes, wherein the electrodes tips are in a non-planar configuration, or are adjustable to a non-planar configuration.

49. A multi-electrode array (MEA) as claimed in claim 48 wherein the electrodes have substantially matched electrical impedance.

50. A method of monitoring electrical signals from a layer of muscle tissue, the method employing a MEA as claimed in claim 48.
